(19) United States
(12) Patent Application Publication
(10) Pub. No.: US 2013/0336928 A1
(43) Pub. Date: Dec. 19, 2013

(54) METHODS AND PHARMACEUTICAL COMPOSITIONS FOR TREATMENT OF CENTRAL NERVOUS SYSTEM DISORDERS WITH THERAPEUTIC AGENT(S) IN PATIENTS WITH CONCURRENT NON-CNS CONDITION(S) OR DISORDERS CONTRAINDICATING SYSTEMIC ADMINISTRATION OF THE THERAPEUTIC AGENT(S)

(71) Applicant: HealthPartners Research & Education, Minneapolis, MN (US)

(72) Inventors: Jacob F. Kobylecky, Portland, OR (US); Benjamin B. Frey, St. Paul, MN (US); William H. Frey, II, White Bear Lake, MN (US)

(21) Appl. No.: 13/916,867

(22) Filed: Jun. 13, 2013

Related U.S. Application Data
(60) Provisional application No. 61/696,434, filed on Sep. 4, 2012.
(60) Provisional application No. 61/661,025, filed on Jun. 18, 2012.

(51) Int. Cl. A61K 9/00

(52) U.S. Cl. CPC A61K 9/0043
USPC 424/85.4; 514/777; 424/94.61; 514/773; 514/784; 514/786

(57) ABSTRACT

Methods for delivering therapeutic agent(s) to the central nervous system to treat a first CNS-related disease and/or condition while avoiding systemic exposure for patients having a second concurrent non-CNS-related disease and/or condition wherein the presence of the second disease and/or condition contraindicates systemic administration of the therapeutic agent(s) and/or pharmaceutical compound to treat the first disease or condition. The present invention provides these advantages by applying the therapeutic agent(s) and/or pharmaceutical composition(s) to the upper third of the nasal cavity, thereby bypassing the blood-brain barrier and administering the therapeutic agent(s) and/or pharmaceutical compound(s) directly to the CNS. This administration method thus protects the mammalian patient, specifically the second non-CNS-related disease and/or condition, by minimizing systemic exposure to the therapeutic agent(s) and/or pharmaceutical compound(s) administered to treat the first disease and/or condition of the CNS.

### CROSS-REFERENCE TO RELATED APPLICATIONS

This application claims the benefit of provisional application No. 61/661,025 filed Jun. 18, 2012 entitled “Treatment of Central Nervous System Disorders in Patients with Concurrent Condition(s) Contraindicating Systemic Administration of Therapeutic Agents”, and further claims the benefit of provisional application No. 61/696,434 filed Sep. 4, 2012 entitled “Treatment of Central Nervous System Disorders in Patients with Concurrent Condition(s) Contraindicating Systemic Administration of Therapeutic Agents”; the entire contents of each of which are hereby incorporated by reference.

### FEDERAL FUNDING

None

### BACKGROUND OF THE INVENTION

1. Field of the Invention

The present invention is directed to methods and pharmaceutical compositions for administering at least one therapeutic agent to the upper third of the nasal cavity of a mammal, thereby enabling the therapeutic agent(s) to bypass the blood-brain barrier to prevent and/or treat a first disease or condition within a mammal's damaged and/or degenerating and/or injured central nervous system, wherein the mammal has a second and concurrent systemic disease or condition that contraindicates systemic administration of the at least one therapeutic agent to treat the CNS-related disease or condition.

2. Description of the Related Art

Many neurological conditions result from damage, degeneration or injury to the central nervous system through aging, disease or injury processes.

The neurological conditions, diseases and/or injuries resulting in damaged and/or degenerating CNS, i.e., cell death, comprise Alzheimer's disease, mild cognitive impairment, age-associated memory impairment, Parkinson's disease, cerebrovascular disease including stroke, Creutzfeldt-Jakob disease, familial amyotrophic lateral sclerosis, lewy-body dementia, atherosclerosis, schizophrenia, autism, tardive dyskinesia, multiple sclerosis, seizure disorders, psychiatric disorders, Wilson's disease, progressive supranuclear palsy, Hallervorden-Spatz syndrome, multisystem atrophy, Huntington's disease, familial basal ganglia degeneration,

Down's syndrome, cataracts, haemochromatosis, thalassemia, cerebral hemorrhage, subarachnoic hemorrhage, head injury, and spinal cord injury. Moreover, certain medical procedures, for example coronary artery bypass graft (CABG) surgery, are associated with neurological complications that result in damage and/or degeneration of the central nervous system and concomitant cell death. In the case of CABG, the surgery is performed on more than 800,000 patients worldwide each year. Many of the CABG procedures performed are associated with neurological complications. These complications range from stroke in up to 16% of the patients to general cognitive decline with 50% of patients having impairment post-surgery and with progressive decline occurring in some patients over the next five years. In addition, physical and behavioral impairment manifest in some CABG patients. Newman M F et al., N. Eng. J. Med. 344:395-402 (2001); Brillman J., Neurol. Clin. 11:475-495 (1993); and Seines, 0. A., Ann. Thorac. Surg. 67:1669-1676 (1999) are instructive.

Administration or application of an effective amount, or therapeutic dose, of certain therapeutic agent(s) and/or pharmaceutical compositions to the upper one-third of a mammalian patient's nasal cavity is a means to bypass the patient's blood-brain barrier (BBB) and actually deliver the effective amount, or therapeutic dose, of the administered/applied agent(s) and/or composition(s) directly to the patient's central nervous system.

Delivery of the agent and/or composition to the upper one third of the patient's nasal cavity is a means of bypassing the BBB to administer therapeutic compounds and/or agents directly to the CNS. Evidence exists that intranasal treatment with certain therapeutic agent(s) improves, i.e., prevents and/or treats, a variety of neurological and psychiatric disorders, e.g., stroke, in animals. When administered to the upper third of the patient's nasal cavity, the vast majority of the administered therapeutic compounds and/or agents are actually delivered to the CNS from the nasal mucosa, bypass the BBB. This basic methodology is discussed and described in U.S. Pat. No. 5,624,898 to Frey II entitled Method for Administering Neurologic

Agents to the Brain, the entire contents of which are hereby incorporated by reference. This administration technique is a vast improvement over systemic administration methods such as intravenous and oral administration of drugs which generally cannot cross the BBB to reach their targets within the CNS. In addition, Frey's intranasal method is a significant improvement over the general inhalation methods which target the lower two-thirds of the patient's nasal cavity. Both the systemic and general intranasal method targeting the lower two-thirds of the nasal cavity result in a very large, unwanted and potentially dangerous systemic exposure to the administered drug or therapeutic agent(s). The present invention addresses, inter alia, this general intranasal problem as well as ensures that the patient's non-CNS, systemic disease and/or condition is protected from exposure to the therapeutic agent administered to the upper third of the nasal cavity, and potential harm therefrom.

General inhalation methods to the lower two-thirds of the nasal cavity delivered by, e.g., nasal spray bottles, on the other hand, result in a large amount of systemic absorption and exposure, with a very small amount, i.e., less than 5%, making the tortuous journey around the turbinates to the upper third of the nasal cavity and further bypassing the BBB to reach the CNS.

Delivery and administration to the upper third of the nasal cavity, is very effective in administering the subject compounds or agents to the desired target, i.e., the CNS, without significant systemic exposure, though some systemic exposure does occur as is further discussed below.

Unwanted systemic exposure of therapeutics used to treat CNS diseases create several serious problems. The systemic metabolism greatly reduces the bioavailability of any agent and/or compound exposed to the non-CNS system. This reduction of bioavailability is increased by unwanted plasma protein binding of the agent and/or compound. As a result, only a small amount of the active therapeutic agent and/or compound actually reaches the CNS. Because of these, inter alia, issues, the actual dose that must be administered in order to achieve a therapeutic dose in the targeted CNS is far larger than the therapeutic dosing. As a consequence, a relatively large concentration of the agent(s) and/or compounds(s) is in the system and will affect non-target systemic organs and systems. This can create unwanted and often dangerous side effects on these non-target organs and systems, particularly in the specific case of patient's having a systemic, non-CNS disorder or condition that contraindicates the systemic use or exposure of the therapeutic agent(s) needed to treat a CNS-related disorder or condition.

We have addressed the efficiency needs in patent application Ser. No. 12/134,385 to Frey II, et al., entitled “Pharmaceutical Compositions and Methods for Enhancing Targeting of Therapeutic Compounds to the Central Nervous System, the entire contents of which are hereby incorporated by reference, and wherein a vasoconstrictor is administered to the patient's nasal cavity either just prior to, or in combination with, administration of at least one therapeutic agent and/or pharmaceutical composition(s) comprising a therapeutic compound(s) and/or agent(s). The efficiency of the direct administration of the pharmaceutical compound to the CNS, with concomitant reduction of systemic exposure of the pharmaceutical compound is remarkable.

Moreover, we provide disclosure of the following patents and applications, each of which are commonly assigned with the present application and incorporated herein in their entirety for disclosure of, inter alia, the various diseases, conditions or disorders of the CNS relating herein to the first disease or condition of the present invention, as well as various compounds and/or therapeutic agents for treating same by application to the upper ⅓ of the nasal cavity, bypassing of the blood-brain barrier and subsequent direct delivery of the compounds and/or agents to the CNS:

U.S. Pat. No. 7,972,595 Methods and compositions for protecting and treating at least one muscarinic receptor from dysfunction not resulting from oxidative stress, toxic actions of metals or infectious agents by administering a pyrophosphate analog;

U.S. Pat. No. 7,786,166 Methods and compositions for protecting and treating muscarinic receptors through administration of at least one protective agent;

U.S. Pat. No. 7,776,312 Method of treating Alzheimer's disease comprising administering deferoxamine (DFO) to the upper one-third of the nasal cavity;

U.S. Pat. No. 7,618,615 Methods for providing neuroprotection for the animal central nervous system against neurodegeneration caused by ischemia;

U.S. Pat. No. 7,084,126 Methods and compositions for enhancing cellular function through protection of tissue components;

U.S. Pat. No. 6,313,093 Method for Administering Insulin to the Brain;

US Pat Application 20100061959 Methods for Providing Neuroprotecton for the Animal Central Nervous System Against the Effects of Ischemia, Neurodegeneration, Trauma, and Metal Poisoning;

US Patent Application 20080305077 Pharmaceutical Compositions and Method for Enhancing Targeting of Therapeutic Compounds to the Central Nervous System;

US Patent Application 20110311654 Methods and Pharmaceutical Compositions for Treating the Animal Central Nervous System for Psychiatric Disorders;

US Patent Application 20110236365 Method for Protecting and Treating at Least One Muscarinic Receptor From Dysfunction Resulting From Free Radical Damage.

There are a number of therapeutic agent(s) and pharmaceutical compositions for treating central nervous system (CNS) diseases, disorders or conditions that are not prescribed to the patient because the patient has a concurrent non-CNS disease or condition, i.e., a systemic disease or condition, that contraindicates the CNS therapeutic agent(s) and/or pharmaceutical composition.

For example and without limitation, it is known that mothers can transmit to their fetuses and babies (via breast milk) nutrients as well as substances detrimental to the health of the fetus and baby. It is, e.g., well known that mothers who drink too much can give birth to children with fetal alcohol syndrome, mothers who smoke too much can give birth to children with low birth weight, certain birth defects, premature birth, and mothers who inhale crack cocaine often give birth to children addicted to crack.

Thus, a number of therapeutic agent(s) and/or pharmaceutical compounds or compositions available for treatment of CNS diseases or disorders cannot be prescribed to pregnant women, women trying to get pregnant or at risk for getting pregnant, or to nursing mothers because of the risk these agent(s) and/or compounds or compositions present to the pregnant mother and/or to the developing fetus.

Additional non-CNS-related diseases and/or conditions exist that contraindicate systemically administered therapeutic agent(s) and/or pharmaceutical compounds to treat CNS-related diseases and/or conditions, for example, young children or the elderly who cannot take oral medication. Patients with damage to the GI tract, including but not limited to throat, stomach or intestinal cancers may also contraindicate systemically administered medications for CNS-related diseases and/or conditions. Patients with liver damage from, inter alia, Hepatitis C, substance abuse such as alcohol or liver cancer also contraindicate systemically administered medications for concurrent CNS-related diseases. Such contraindicated systemic, or non-CNS diseases and/or conditions are discussed further infra.

Thus, it would be advantageous to deliver therapeutic agent(s) to the damaged, degenerating and/or injured central nervous system to treat a first CNS-related disease and/or condition in such a way as to avoid systemic exposure for patients having a second and concurrent non-CNS-related disease and/or condition wherein the presence of the second disease and/or condition contraindicates systemic administration of the therapeutic agent(s) and/or pharmaceutical compound to treat the first disease or condition.

The present invention provides these advantages by applying the therapeutic agent(s) and/or pharmaceutical composition(s) to the upper third of the nasal cavity, thereby bypassing the blood-brain barrier and administering the therapeutic agent(s) and/or pharmaceutical compound(s) directly to the CNS. This administration method thus protects the mammalian patient, specifically the patient's second, concurrent non-CNS-related disease and/or condition, by minimizing systemic exposure to the therapeutic agent(s) and/or pharmaceutical compound(s) administered to treat the first disease and/or condition of the CNS.

The present invention provides solutions for, inter alia, these problems.

### SUMMARY OF THE INVENTION

Given the situation described above there is a need for a method for efficient and non-invasive delivery of at least one therapeutic agent and/or pharmaceutical composition directly to the damaged and/or degenerating central nervous system of a mammalian patient for treatment of a first CNS-related disease and/or condition wherein the a mammalian patient wherein the patient has a second non-CNS-related disease and/or condition that contraindicates systemic administration of the therapeutic agent(s) and/or pharmaceutical composition(s) used to treat the first CNS disease and/or condition.

### DETAILED DESCRIPTION OF THE INVENTION

### Definitions

As used herein, “central nervous system” (CNS) refers to the brain and spinal cord and associated cells and tissues.

As used herein, a “first disease or condition of a mammal's damaged or degenerating or injured central nervous system” refers to central nervous system diseases, disorders and/or conditions such as brain diseases and conditions that comprise ischemia, i.e., cerebral ischemia, ischemia, stroke, neurodegeneration, neurological complications arising from such as Alzheimer's disease, Parkinson's disease, Wilson's disease, Lewy body dementia, multiple sclerosis, seizure disorders including but not limited to epilepsy, cerebellar ataxia, progressive supranuclear palsy, amyotrophic lateral sclerosis, autism, affective disorders, anxiety disorders, psychiatric disorders generally, depression, schizophrenia, post-traumatic stress disorder, metabolic disorders that affect the CNS; cell damage, nerve damage from cerebrovascular disorders such as stroke in the brain or spinal cord, from CNS infections including meningitis and HIV/AIDS, from tumors of the brain and spinal cord, prion diseases, and CNS disorders resulting from ordinary aging (e.g., anosmia), head and/or brain injury, or spinal cord injury, and any other medical diseases and conditions mentioned herein with neurodegeneration, neurological cell loss and/or damage or degeneration.

As used herein, a “second systemic, non-CNS disease or condition” refers to any disease, disorder and/or condition that is not within the patient's CNS and which is concurrently present within a patient having the first disease or condition defined supra, and which contraindicates systemic administration and treatment of the concurrent first disease or condition of the patient's CNS.

As used herein, “systemic administration” refers to administration of a medication, pharmaceutical and the like by the following non-limited means: oral, intravenous, intra-arterial, intramuscular, epidermal, transdermal, subcutaneous, topic, sublingual as well as general inhalation, i.e., administration to the lower two-thirds of the patient's nasal cavity. In each of these cases, the administered drug will migrate through the patient's circulatory system and, in order to reach the patient's CNS would be required to cross the patient's blood-brain barrier.

In the context of the present invention, the terms “treat” and “therapy” and the like refer to alleviate, slow the progression, prophylaxis, attenuation or cure of existing disease or condition that has or is causing cell death in the CNS. “Prevent”, as used herein, refers to putting off, delaying, slowing, inhibiting, or otherwise stopping, reducing or ameliorating the onset of such diseases or disorders. It is preferred that a large enough quantity of the therapeutic agent(s) and/or compound(s) be applied in non-toxic levels in order to provide an effective level of activity against the disease. The method of the present invention may be used with any animal, such as a mammal or a bird (avian), more preferably a mammal. Poultry are a preferred bird. Exemplary mammals include, but are not limited to rats, mice, cats, dogs, horses, cows, sheep, pigs, and more preferably humans.

“Ischemia” or ischemic episode or condition is defined herein to comprise an ischemic condition where the brain or parts of the brain do not receive enough blood flow to maintain normal neurological function, resulting in a loss or death of CNS cells and concomitant damage and/or degeneration of the CNS tissues. Various conditions and/or diseases can cause ischemia, including but not limited to stroke. Some of the neurological disorders and diseases of the CNS defined and discussed herein are characterized by some level of ischemia. The neurological disorders and diseases of the CNS defined and discussed herein are amenable to treatment with the present invention.

An “effective amount” of therapeutic agent(s) and/or component(s) of the pharmaceutical composition of the present invention comprising therapeutic agent(s) is an amount sufficient to prevent, treat, reduce and/or ameliorate the symptoms, neuronal damage and/or underlying causes of any of the referenced disorders or diseases. In some instances, an “effective amount” is sufficient to eliminate the symptoms of those diseases and overcome the disease itself. Preferably, an effective amount of the at least one therapeutic agent and/or component(s) of the pharmaceutical composition yields a tissue concentration in the range of about 10⁻⁷ molar to about 10⁻⁵ molar, but the concentrations may be greater provided that toxicity is avoided.

For illustrative purposes only, exemplary treatment regimens relating generally to the therapeutic agent(s) and/or pharmaceutical compounds disclosed herein, including dosage ranges, volumes and frequency are provided below: Efficacious dosage range for the at least one therapeutic agent, delivery-enhancement agents, vasoconstrictors and/or antibiotics comprises 0.0001-1.0 mg/kg.

A more preferred dosage range may be 0.005-1.0 mg/kg.
The most preferred dosage range may be 0.05-1.0 mg/kg.
The dosage volume (applicable to nasal sprays or drops) range may be 0.015 m1-1.0 ml.
The preferred dosage volume (applicable to nasal sprays or drops) range may be 0.03 m1-0.6 ml.

The brain concentrations that are likely to be achieved with the dosage ranges provided above are, for a single dose: 0.1 nM-5 μM. Over the course of a multi-dose treatment plan, the maximum brain concentration may be as high as 50 μM for deliver-enhancement agents and antibiotics.

The present invention therefore provides methods therapies used to non-invasively treat CNS disorders without harming the patient in terms of a second systemic, non-CNS condition that the patient presents by minimizing exposure of the patient's non-CNS system, and the second condition therein, to a therapeutic agent(s) and/or pharmaceutical compound(s) administered to treat the CNS disease or condition.

Exemplary second systemic diseases or conditions according to the present invention comprise and without limitation pregnant women, women trying to get pregnant or at risk for getting pregnant, or to nursing mothers because of the risk these agent(s) and/or compounds or compositions present to the pregnant mother and/or to the developing fetus.

Additional non-CNS-related diseases and/or conditions exist that contraindicate systemically administered therapeutic agent(s) and/or pharmaceutical compounds to treat CNS-related diseases and/or conditions, for example, young children or the elderly who cannot take oral medication. Patients with damage to the GI tract, including but not limited to throat, stomach or intestinal cancers may also contraindicate systemically administered medications for CNS-related diseases and/or conditions. Further, patients with liver damage due, e.g., Hepatitis C, substance abuse such as alcohol, and liver cancer may also contraindicate systemic administration of therapeutic agent(s) for treatment of a concurrent CNS-related disorder.

Consequently, a number of therapeutic agent(s) and/or pharmaceutical compounds or compositions available for treatment of CNS diseases or disorders are contraindicated for use in treating patients for at least one second, concurrent and non-CNS and systemic disease or disorder or condition that contraindicate the systemic administration of the agent(s) and/or composition(s) needed for treating the CNS diseases or disorders.

Exemplary combinations of first and second diseases under the present invention may comprise:

(1) A female mammalian patient with a brain injury, stroke, and/or epilepsy (first CNS-related disease or condition) and who also is either pregnant, at risk of becoming pregnant (due to no birth control use) or is breast feeding (second non-CNS disease or condition that contraindicates systemic administration of therapeutic agent(s) to treat the brain injury, ischemia and/or epilepsy for fear of injuring the female, the fetus or the nursing infant, respectively);

(2) A mammalian patient with Parkinson's disease (first CNS-related disease or condition) who also has liver damage due to Hepatitis C, alcoholism, cancer or other ailment (second non-CNS disease or condition that contraindicates systemic administration of Parkinson's medication);

(3) An elderly mammalian patient with Alzheimer's disease (first CNS-related disease or condition) who also has liver damage as in example 2;

(4) A female mammalian patient with depression (first CNS-related disease or condition) and who also is either pregnant, at risk of becoming pregnant (due to no birth control use) or is breast feeding (second non-CNS disease or condition that contraindicates systemic administration of therapeutic agent(s) to treat the depression for fear of injuring the female, the fetus or the nursing infant, respectively);

(5) A mammalian patient with post-traumatic stress disorder (PTSD) (first CNS-related disease or condition) and who also is an alcoholic with liver damage as a result (second non-CNS disease or contraindicates systemic administration of therapeutic agent(s) to treat the PTSD).

Many other exemplary combinations of a first CNS-related disease and/or condition in combination with a second non-CNS, systemic disease and/or condition that contraindicates systemic treatment of the first disease and/or condition will present themselves now to the skilled artisan. The present invention solves this problem effectively, efficiently and non-invasively.

### Transportation Pathway to Bypass Blood-Brain Barrier

### The Olfactory Nerve

Various methods of the present invention include administration of the therapeutic agent(s) and/or pharmaceutical composition(s) of the present invention to tissue innervated by the olfactory nerve and that is located in the upper third of the nasal cavity. The therapeutic agent(s) and/or pharmaceutical composition(s) of the present invention can be delivered to the olfactory area via application to the upper third of the nasal cavity.

Fibers of the olfactory nerve are unmyelinated axons of olfactory receptor cells that are located in the upper one-third of the nasal mucosa. The olfactory receptor cells are bipolar neurons with swellings covered by hair-like cilia that project into the nasal cavity. At the other end, axons from these cells collect into aggregates and enter the cranial cavity at the roof of the nose. Surrounded by a thin tube of pia, the olfactory nerves cross the subarachnoid space containing CSF and enter the inferior aspects of the olfactory bulbs. Once the therapeutic agent(s) and/or pharmaceutical composition(s) of the present invention is applied to the upper third of nasal cavity, the therapeutic agent(s) and/or pharmaceutical composition(s) of the present invention can undergo transport through the nasal mucosa and into the olfactory bulb and other areas of the CNS, such as the anterior olfactory nucleus, frontal cortex, hippocampal formation, amygdaloid nuclei, nucleus basalis of Meynert, hypothalamus, midbrain, cerebellum, cervical spinal cord and the like.

### Neuronal Transport

Embodiments of the present method includes administration of the therapeutic agent(s) and/or pharmaceutical composition(s) of the present invention to the subject by application to the upper third of the mammalian subject's nasal cavity. Application of the therapeutic agent(s) and/or pharmaceutical composition(s) of the present invention in this manner ensures that the therapeutic agent(s) and/or pharmaceutical composition(s) are transported to the CNS, brain, and/or spinal cord along a neural pathway, with reduced systemic loss and, therefore, minimized systemic exposure. A neural pathway includes transport within or along a neuron, through or by way of lymphatics running with a neuron, through or by way of a perivascular space of a blood vessel running with a neuron or neural pathway, through or by way of an adventitia of a blood vessel running with a neuron or neural pathway, or through an hemangiolymphatic system.

The present invention comprises transportation of the therapeutic agent(s) and/or pharmaceutical composition(s) by way of a neural pathway, rather than through the circulatory system, so that agent(s) and/or compound(s) that are unable to, or only poorly, cross the blood-brain barrier from the bloodstream into the brain can be delivered to the lymphatic system, CNS, brain, and/or spinal cord. The therapeutic agent(s) and/or pharmaceutical composition(s) of the present invention, once past the blood-brain barrier and in the CNS, can then be delivered to various areas of the brain or spinal cord through lymphatic channels, through a perivascular space, or transport through or along neurons.

Use of a neural pathway to transport a therapeutic agent(s) and/or pharmaceutical composition(s) to the brain, spinal cord, or other components of the central nervous system obviates the obstacle presented by the blood-brain barrier so that medications, i.e., therapeutic agent(s) and/or pharmaceutical compositions of the present invention, that cannot normally cross that barrier, can be delivered directly to the CNS, e.g., the brain and spinal cord. In addition, the present invention can provide for delivery of a more concentrated level of the therapeutic agent(s) and/or pharmaceutical composition(s) of the present invention to the CNS since the therapeutic agent(s) and/or pharmaceutical composition(s) of the present invention do not become diluted in fluids present in the bloodstream. As such, the invention provides an improved method for delivering an effective amount or therapeutic dose of the therapeutic agent(s) and/or pharmaceutical composition(s) of the present invention directly to the CNS including the brain and/or spinal cord, and avoids exposing the second, concurrent non-CNS disorder or condition of a patient to the therapeutic agent(s) administered for treatment of the first CNS disorder or condition.

### The Olfactory Neural Pathway

One embodiment of the present method includes delivery of the therapeutic agent(s) to the subject in a manner such that the therapeutic agent(s) and/or pharmaceutical compound(s) is transported into the CNS, e.g., the brain, and/or spinal cord along an olfactory neural pathway. Typically, such an embodiment includes administering the agent(s) and/or compound(s) to tissue innervated by the olfactory nerve and inside the nasal cavity. The olfactory neural pathway innervates primarily the olfactory epithelium in the upper third of the nasal cavity, as described above. Application of the agent(s) and/or compound(s) to a tissue innervated by the olfactory nerve can deliver the agent(s) and/or compound(s) to damaged neurons or cells of the CNS, including but not limited to the brain, and/or spinal cord. Olfactory neurons innervate this tissue and can provide a direct connection to the CNS, brain, and/or spinal cord due, it is believed, to their role in olfaction.

Delivery through the olfactory neural pathway can employ lymphatics that travel with the olfactory nerve to the various brain areas and from there into dural lymphatics associated with portions of the CNS, such as the spinal cord. Transport along the olfactory nerve can also deliver therapeutic agent(s) and/or compound(s) to an olfactory bulb. A perivascular pathway and/or a hemangiolymphatic pathway, such as lymphatic channels running within the adventitia of cerebral blood vessels, can provide an additional mechanism for transport of therapeutic agents to the brain and spinal cord from tissue innervated by the olfactory nerve.

Therapeutic agent(s) and/or pharmaceutical compositions thereof may be administered to the olfactory nerve, for example, through the olfactory epithelium located at the upper one-third of the nasal cavity. Such administration can employ extracellular or intracellular (e.g., transneuronal) anterograde and retrograde transport of the agent(s) and/or compound(s) entering through the olfactory nerves to the brain and its meninges, to the brain stem, or to the spinal cord. Once the effective amount, i.e., therapeutic dose, of the therapeutic agent(s) and/or pharmaceutical composition thereof is dispensed into or onto tissue innervated by the olfactory nerve, the therapeutic agent(s) and/or pharmaceutical composition and/or components thereof may be transported through the tissue and travel along olfactory neurons into areas of the CNS including but not limited to the brain stem, cerebellum, spinal cord, cerebrospinal fluid, olfactory bulb, and cortical and subcortical structures. The blood-brain barrier is bypassed in the present invention by application of an effective amount of the therapeutic agent(s) and/or pharmaceutical composition(s) comprising therapeutic agent(s) and/or composition(s) or compound(s) to the upper third of the nasal cavity of the patient, e.g., a mammal. The effective amount of the therapeutic agent(s) and/or pharmaceutical composition of the invention migrate from the nasal mucosa through foramina in the cribriform plate along the olfactory neural pathway and is delivered directly into the CNS. Further, vasoconstrictors may be applied to the nasal cavity of the patient, either before or during the application of the therapeutic agent(s) and/or pharmaceutical composition(s) to the upper third of the patient's nasal cavity, to enhance the efficiency of delivery of the agent(s) to the patient's CNS and minimization of any potential systemic exposure of the therapeutic agent(s).

Administration to the nasal cavity employing a neural pathway can thus deliver therapeutic agent(s) and/or pharmaceutical compositions to the lymphatic system, brain stem, cerebellum, spinal cord, and cortical and subcortical structures of the mammalian patient. The therapeutic agent(s) and/or pharmaceutical composition of the present invention alone may facilitate this movement into the CNS, i.e., brain, and/or spinal cord. Alternatively, a carrier and/or the delivery-enhancement agent(s) may assist in the transport of the therapeutic agent(s) and/or pharmaceutical composition of the present invention into and along the neural pathway. Administration of the therapeutic agent(s) and/or pharmaceutical composition(s) of the present invention to the upper third of the mammalian patient's nasal cavity thus bypasses the blood-brain barrier through a transport system from the nasal mucosa and/or epithelium to the CNS, i.e., brain and spinal cord.

Various embodiments of the invention administer the therapeutic agent(s) and/or pharmaceutical composition(s) of the present invention to tissue innervated by the olfactory nerves. Such nerve systems can provide a direct connection between the outside environment and the brain, thus providing advantageous delivery of the agent(s) and/or compound(s) to the CNS, including brain, brain stem, and/or spinal cord. The therapeutic agent(s) and/or pharmaceutical composition(s) of the present invention may be unable to cross or inefficiently cross the blood-brain barrier from the bloodstream into the brain. Alternatively, for those agent(s) and/or composition(s) that may cross the blood-brain barrier, the present invention offers an alternative treatment for those patients having a concurrent system, non-CNS disease or condition that contraindicates systemic administration of the therapeutic agent(s) and/or compositions(s) needed within the CNS to treat a first CNS-related disease, condition or disorder. Thus, the methods of the present invention allow for the delivery of the therapeutic agent(s) and/or pharmaceutical composition(s) by way of the olfactory nerve rather than through the circulatory system in order to facilitate treatment of the patient's CNS-related disorder, disease or condition while protecting the mammalian patient's non-CNS, systemic disorder disease or condition from the administered therapeutic agent/composition intended for delivery to the CNS. Thus, this method of administration allows for the efficient and non-invasive delivery of the therapeutic agent(s) and/or pharmaceutical composition(s) of the present invention to the CNS, brain, or spinal cord without systemic loss or exposure and concomitant protection of the mammalian patient's concurrent non-CNS, systemic disorder, disease or condition.

### Alternative Pathways

Alternative non-systemic pathways to the olfactory nerve pathway discussed above comprise pathways along other nerves that innervate the nasal cavity, e.g., the trigeminal pathway, well known to the skilled artisan.

### Delivery-Enhancement Agents

Certain compounds, i.e., delivery-enhancement agents, may be utilized by the present invention to assist the therapeutic agent(s) in delivery to the central nervous system and the damaged, diseased or injured regions therein while protecting a patient's second, systemic, concurrent non-CNS disorder, disease or condition from exposure to the therapeutic agent(s). A preferred delivery-enhancement agent comprises hyaluronidase which has been observed to very significantly increase delivery of therapeutic agent(s) to the CNS when applied to the upper third of the nasal cavity as either a pretreatment administered in an effective amount prior to the application of therapeutic agent(s) according to one embodiment of the present invention, or as a component of the pharmaceutical composition comprising therapeutic agent(s) of the present invention, or as a separate compound administered intranasally to the upper third of the nasal cavity substantially simultaneously as the therapeutic agent(s) and/or pharmaceutical composition. It is believed that the hyaluronidase acts on hyaluronic acid in the extracellular matrix to enhance delivery of therapeutic agent(s) and/or pharmaceutical compositions to the CNS.

Alternative delivery-enhancement agents comprise neuregulin, migration-inducing activity and leukemia inhibitory factor. These delivery-enhancement agents, e.g., hyaluronidase, lipophilic agents, neuregulin, migration-inducing activity and leukemia inhibitory factor may be used individually, or in any combination, to enhance delivery of the therapeutic agent(s) to the CNS according to the present invention. Therefore, at least one delivery-enhancement agent may be used as a pretreatment to transportation of the therapeutic agent(s) and/or pharmaceutical composition and/or as a component of the pharmaceutical composition comprising therapeutic agent(s).

Alternative delivery-enhancement agents that further enhance the mucosal delivery of therapeutic agent(s) and/or pharmaceutical composition comprising therapeutic agent(s) of the present invention, comprise an enzyme inhibitor, particularly proteases inhibitors as is well known to those in the art. Protease inhibitors may include, but are limited to, antipain, arphamenine A and B, benzamidine HCl, AEBSF, CA-074, calpain inhibitor I and II, calpeptin, pepstatin A, actinonin, amastatin, bestatin, boroleucine, captopril, chloroacetyl-HOLeu-Ala-Gly-NH2, DAPT, diprotin A and B, ebelactone A and B, foroxymithine, leupeptin, pepstatin A, phosphoramidon, aprotinin, puromycin, BBI, soybean trypsin inhibitor, phenylmethylsulfonyl fluoride, E-64, chymostatin, 1,10-phenanthroline, EDTA and EGTA.

Still further alternative delivery-enhancement agents may include, but are not limited to, surfactants, bile salts, dihydrofusidates, bioadhesive agents, phospholipid additives, mixed micelles, liposomes, or carriers, alcohols, enamines, cationic polymers, NO donor compounds, long-chain amphipathic molecules, small hydrophobic penetration enhancers; sodium or a salicylic acid derivatives, glycerol esters of acetoacetic acid, cyclodextrin or beta-cyclodextrin derivatives, medium-chain fatty acids, chelating agents, amino acids or salts thereof, N-acetylamino acids or salts thereof, mucolytic agents, enzymes specifically targeted to a selected membrane component, inhibitors of fatty acid synthesis and inhibitors of cholesterol synthesis. The present invention contemplates using one or more, i.e., at least one, of the above delivery-enhancement agents, either alone or in combination with the therapeutic agent(s) comprising a pharmaceutical compound in an effective amount.

interferons (i.e., IFN-alpha, IFN-beta, IFN-gamma, IFN-omega, and IFN-tau); and any biologically active variants thereof.

### Antibiotic Agents

In various embodiments, the present invention may further comprise an effective amount of at least one antibiotic, or alternatively at least one antibiotic(s) pretreatment administered prior to application of the therapeutic agent(s) and/or pharmaceutical composition to the upper third of the mammalian patient's nasal cavity may be used, or any combination thereof, to protect the patient undergoing treatment from nasal bacteria migrating along the neural pathway followed by the therapeutic agent(s) and/or pharmaceutical composition(s) according to the present invention. Further, the antibiotic(s) may be delivered as a pretreatment, co-treatment and/or post treatment systemically and/or by application to the upper third of the nasal cavity. The utility of such an antibiotic element within the present invention is to reduce the risk that bacteria found in the nasal cavity may enter the nasal tissues at the upper third of the nasal cavity during application of the therapeutic agent(s) and/or pharmaceutical composition, cross the blood-brain barrier and infect other tissues within the CNS. Particular tissues of concern include, but are not limited to, the brain, meninges, blood, spinal cord, and other peripheral tissues. A preferred embodiment is to pretreat and/or simultaneously treat the patient with antibiotic(s) when the delivery-enhancement agent(s), e.g., hyaluronidase, is applied to the upper third of the nasal cavity.

For example, in one study, mupirocin smeared inside the nose cut infection rates in half or better Staphylococcus aureus is a widely distributed germ that normally resides in the nostrils of an estimated 25 to 30 percent of all hospitalized patients without causing harm. But this bacteria can contaminate surgical sites, causing severe and often deadly infections, especially in people with weakened immune systems.

Another study found that nasal xylitol, an over the counter remedy sold in health food stores, can reduce nasal bacteria and their ability to hold onto and infect cells in the nasal mucosa. Still other studies have found that defensins, a natural antibiotic found in mucosa in the human, can protect against bacterial infection and enhance immune protective function. Mammalian defensins are small, cationic, antimicrobial peptides encoded by the host that are considered to be important antibiotic-like effectors of innate immunity. By using chemokine receptors on dendritic cells and T cells, defensins might also contribute to the regulation of host adaptive immunity against microbial invasion. Defensins have considerable immunological adjuvant activity and linkage of beta-defensins or selected chemokines to an idiotypic lymphoma antigen has yielded potent antitumor vaccines. The functional overlap between defensins and chemokines is reinforced by reports that some chemokines have antimicrobial activities. Although showing similarity in activity and overall tertiary structure, the evolutionary relationship between defensins and chemokines remains to be determined. (De Yang, et al., Mammalian defensins in immunity: more than just microbicidal. Trends lmmunol. 2002 Jun ;23 (6):291-6 12072367).

Exemplary antibiotics for use in the present invention comprise mupirocin, defensin, gentamycin, geneticin, cefminoxime, penicillin, streptomycin, xylitol, or other antibiotic, either alone or in combination to assist in protecting the patient who is receiving therapeutic agent(s) and/or pharmaceutical composition of the present invention. The use of such antibiotics within nasal treatments is widely reported in the literature as will be readily recognized by the skilled artisan, however no such nasal treatment is reported in conjunction with the intranasal application of therapeutic agent(s) and/or pharmaceutical compositions to the upper third of the nasal cavity whereby the blood-brain barrier is bypassed and wherein the subject mammalian patient further comprises a second non-CNS, systemic disease, disorder or condition that contraindicates the systemic administration of the therapeutic agent(s) and/or pharmaceutical composition applied to the upper third of the nasal cavity for direct delivery to the CNS and treatment of diseases, disorders and conditions therein.

### Pharmaceutical Composition

In addition to the effective amount of at least one therapeutic agent administered to the upper third of the mammalian nasal cavity, a pharmaceutical composition may be applied or administered to the upper third of the nasal cavity in accordance with the present invention. Such a pharmaceutical composition may comprise, in addition to the effective amount of at least one therapeutic agent, for example, the composition can comprise at least one vasoconstrictor as described supra, at least one delivery-enhancement agent as described supra, and/or at least one antibiotic, all as described supra. The pharmaceutical composition of certain embodiments of the present invention may be combined with pre-, co-, and post-treatment with any combination of systemic and/or application to the upper third of the nasal cavity of the at least one vasoconstrictor, delivery-enhancement agent, and/or antibiotic.

Among the alternatives that may be combined with therapeutic agent(s) in the pharmaceutical composition are delivery-enhancement agents, such as lipophilic agents, that can enhance absorption of the therapeutic agent(s) through the mucosa or epithelium of the nasal cavity to reach damaged and/or degenerating and/or injured regions within in the CNS. Among the preferred lipophilic substances are cationic liposomes including one or more of phosphatidyl choline, lipofectin, DOTAP, or the like.

A preferred delivery-enhancement agent comprises hyaluronidase which has been observed to very significantly increase delivery of therapeutic agent(s) to the CNS when applied to the upper third of the nasal cavity as either a pretreatment to the therapeutic agent(s) application of the present invention, or as a component of a pharmaceutical composition comprising therapeutic agent(s) of the present invention. Alternative delivery-enhancement agents comprise neuregulin and migration-inducing activity. These delivery-enhancement agents, e.g., hyaluronidase, lipophilic agents, neuregulin and migration-inducing activity may be used individually, or in any combination, to enhance delivery of the therapeutic agent(s) to the CNS according to the present invention. Therefore, at least one delivery-enhancement agent may be used as a pretreatment to transportation of the therapeutic cells and/or pharmaceutical composition and/or as a component of the pharmaceutical composition comprising therapeutic agent(s).

The pharmaceutical composition of the present invention may further comprise at least one antibiotic, or alternatively an antibiotic pretreatment prior to application of the pharmaceutical composition to the upper third of the nasal cavity may be used, or any combination thereof, to protect the patient undergoing therapeutic agent therapy of the present invention. Further, the antibiotic may be delivered as a pretreatment, co-treatment and/or post treatment given intranasally and/or systemically. The utility of such an antibiotic element within the present invention is to reduce the risk that bacteria found in the nasal cavity may enter the nasal tissues at the upper third of the nasal cavity during application of the therapeutic agent(s) and/or pharmaceutical composition, cross the blood-brain barrier and infect other tissues within the CNS. Particular tissues of concern include, but are not limited to, the brain, meninges, blood, spinal cord, and other peripheral tissues. A preferred embodiment is to pretreat and/or simultaneously treat the patient with antibiotic when a delivery-enhancement agent such as hyaluronidase is applied, either alone or in a pharmaceutical composition, to the upper third of the nasal cavity.

Further, the pharmaceutical composition of the present invention may comprise any pharmaceutically acceptable additive, carrier, and/or adjuvant that can promote the transfer of the therapeutic agent(s) within or through a tissue innervated by the trigeminal nerve or olfactory nerve or along or through a neural pathway for delivery to the CNS.

By “pharmaceutically acceptable carrier” is intended a carrier that is conventionally used in the art to facilitate the storage, administration, and/or the biological activity of therapeutic agent(s), delivery-enhancement agent(s), and/or antibiotic(s) within a pharmaceutical composition of the present invention. A carrier may also reduce any undesirable side effects of the components of such a pharmaceutical composition. A suitable carrier should be stable, i.e., incapable of reacting with other ingredients in the formulation. It should not produce significant local or systemic adverse effect in recipients at the dosages and concentrations employed for treatment. Such carriers are generally known in the art.

Suitable carriers for the various embodiments of the present invention include those conventionally used for large stable macromolecules such as albumin, gelatin, collagen, polysaccharide, monosaccharides, polyvinylpyrrolidone, polylactic acid, polyglycolic acid, polymeric amino acids, fixed oils, ethyl oleate, liposomes, glucose, sucrose, lactose, mannose, dextrose, dextran, cellulose, mannitol, sorbitol, polyethylene glycol (PEG), and the like. A further pharmaceutical composition may comprise microparticles, organic and inorganic compounds serving as an adherence material for the agent(s) that may be transported to the CNS in various embodiments of the present invention, thus diminishing the loss of the therapeutic agent(s) transported from the upper third of the patient's nasal cavity to the CNS. These compounds may include several kinds of adhesive molecules, gels (serving as an encapsulating/embedding material for the therapeutic agent(s)), components of extracellular matrix or matrices, and organic and/or inorganic particles such as fibrin or fibronectin carbon-or clay- and dextran particles and their composition.

Water, saline, aqueous dextrose, and glycols are preferred liquid carriers, particularly (when isotonic) for solutions. The carrier can be selected from various oils, including those of petroleum, animal, vegetable or synthetic origin, for example, peanut oil, soybean oil, mineral oil, sesame oil, and the like. Suitable pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol, and the like. The compositions can be subjected to conventional pharmaceutical expedients, such as sterilization, and can contain conventional pharmaceutical additives, such as preservatives, stabilizing agents, wetting, or emulsifying agents, salts for adjusting osmotic pressure, buffers, and the like. Where the carrier is a liquid, it is preferred that the carrier be hypotonic or isotonic with body fluids and have a pH within the range of 4.5-8.5.

Other acceptable components in the pharmaceutical composition comprise, without limitation, isotonicity-modifying agents such as water, saline, and buffers including phosphate, citrate, succinate, acetic acid, and other organic acids or their salts. Typically, the pharmaceutically acceptable carrier also includes one or more stabilizers, reducing agents, anti-oxidants and/or anti-oxidant chelating agents. The use of buffers, stabilizers, reducing agents, anti-oxidants and chelating agents in the preparation of protein-based compositions, particularly pharmaceutical compositions, is well known in the art. See, Wang et al. (1980) J. Parent. Drug Assn. 34(6):452-462; Wang et al.(1988) J. Parent. Sci. Tech. 42:S4-S26 (Supplement); Lachman et al.(1968) Drug and Cosmetic Industry 102(1):36-38, 40, and 146-148; Akers (1988) J. Parent. Sci. Tech. 36(5):222-228; and Methods in Enzymology, Vol. XXV, ed. Colowick and Kaplan, “Reduction of Disulfide Bonds in Proteins with Dithiothreitol,” by Konigsberg, pp. 185-188.

Various embodiments of the pharmaceutical composition of the present invention comprise suitable buffers such as acetate, adipate, benzoate, citrate, lactate, maleate, phosphate, tartarate, borate, tri(hydroxymethyl aminomethane), succinate, glycine, histidine, the salts of various amino acids, or the like, or combinations thereof. See Wang (1980) supra at page 455. Suitable salts and isotonicifiers include sodium chloride, dextrose, mannitol, sucrose, trehalose, or the like.

Various embodiments of the pharmaceutical composition of the present invention may further comprise suitable reducing agents, which maintain the reduction of reduced cysteines, include dithiothreitol (DTT also known as Cleland's reagent) or dithioerythritol at 0.01% to 0.1% wt/wt; acetylcysteine or cysteine at 0.1% to 0.5% (pH 2-3); and thioglycerol at 0.1% to 0.5% (pH 3.5 to 7.0) and glutathione. Suitable antioxidants include sodium bisulfite, sodium sulfite, sodium metabisulfite, sodium thiosulfate, sodium formaldehyde sulfoxylate, and ascorbic acid. Suitable chelating agents, which chelate trace metals to prevent the trace metal catalyzed oxidation of reduced cysteines, include citrate, tartarate, ethylenediaminetetraacetic acid (EDTA) in its disodium, tetrasodium, and calcium disodium salts, and diethylenetriamine pentaacetic acid (DTPA). See, e.g., Wang (1980) supra at pages 457-458 and 460-461, and Akers (1988) supra at pages 224-227.

Various embodiments of the pharmaceutical composition of the present invention may further comprise one or more preservatives such as phenol, cresol, paraaminobenzoic acid, BDSA, sorbitrate, chlorhexidine, benzalkonium chloride, or the like. Suitable stabilizers include carbohydrates such as trehalose or glycerol. The composition can include a stabilizer such as one or more of microcrystalline cellulose, magnesium stearate, mannitol, or sucrose to stabilize, for example, the physical form of the composition; and one or more of glycine, arginine, hydrolyzed collagen, or protease inhibitors to stabilize, for example, the chemical structure of the composition.

Various embodiments of the pharmaceutical composition of the present invention may also comprise suitable suspending agents such as carboxymethyl cellulose, hydroxypropyl methylcellulose, hyaluronic acid, alginate, chondroitin sulfate, dextran, maltodextrin, dextran sulfate, or the like. The composition can include an emulsifier such as polysorbate 20, polysorbate 80, pluronic, triolein, soybean oil, lecithins, squalene and squalanes, sorbitan treioleate, or the like.

The pharmaceutical composition of the present invention may further comprise at least one antimicrobial such as phenylethyl alcohol, phenol, cresol, benzalkonium chloride, phenoxyethanol, chlorhexidine, thimerosol, or the like. Suitable thickeners include natural polysaccharides such as mannans, arabinans, alginate, hyaluronic acid, dextrose, or the like; and synthetic ones like the PEG hydrogels of low molecular weight; and aforementioned suspending agents may be included in the pharmaceutical composition of the present invention.

The inventive pharmaceutical composition may further comprise include an adjuvant such as cetyl trimethyl ammonium bromide, BDSA, cholate, deoxycholate, polysorbate 20 and 80, fusidic acid, or the like. Suitable sugars include glycerol, threose, glucose, galactose, mannitol, and sorbitol.

Various embodiments of the pharmaceutical composition of the present invention may further comprise one or more of a solubility enhancing additive, preferably a cyclodextrin; a hydrophilic additive, preferably a monosaccharide or oligosaccharide; an absorption promoting additive, preferably a cholate, a deoxycholate, a fusidic acid, or a chitosan; a cationic surfactant, preferably a cetyl trimethyl ammonium bromide; a viscosity enhancing additive, preferably to promote residence time of the composition at the site of administration, preferably a carboxymethyl cellulose, a maltodextrin, an alginic acid, a hyaluronic acid, or a chondroitin sulfate; or a sustained release matrix, preferably a polyanhydride, a polyorthoester, a hydrogel, a particulate slow release depo system, preferably a polylactide co-glycolides (PLG), a depo foam, a starch microsphere, or a cellulose derived buccal system; a lipid-based carrier, preferably an emulsion, a liposome, a niosome, or a micelle. The composition can include a bilayer destabilizing additive, preferably a phosphatidyl ethanolamine; a fusogenic additive, preferably a cholesterol hemisuccinate.

These lists of carriers and additives are by no means complete, and a worker skilled in the art can choose excipients from the GRAS (generally regarded as safe) list of chemicals allowed in the pharmaceutical preparations and those that are currently allowed in topical and parenteral formulations.

Moreover, the method for formulating a pharmaceutical composition is generally known in the art. A thorough discussion of formulation and selection of pharmaceutically acceptable carriers, stabilizers, and isomolytes can be found in Remington's Pharmaceutical Sciences (18.sup.th ed.; Mack Publishing Company, Eaton, Pa., 1990), herein incorporated by reference.

For the purposes of this invention, the pharmaceutical composition as described herein can be formulated in a unit dosage and in a form such as a solution, suspension, or emulsion for application to the upper third of the mammalian patients nasal cavity. The pharmaceutical composition to be applied and administered to the upper third of the nasal cavity to the tissue innervated by the olfactory neurons may be in the form of a powder, a granule, a solution, a spray (e.g., an aerosol), an ointment, an infusion, a drop, or a sustained-release composition, such as a polymer disk. Other forms of compositions for administration include a suspension of a particulate, such as an emulsion, a liposome, an insert that releases the pharmaceutical composition slowly, and the like. The powder or granular forms of the pharmaceutical composition may be combined with a solution and with a diluting, dispersing, or surface active regulatory agent. The composition can also be in the form of lyophilized powder, which can be converted into solution, suspension, or emulsion before administration.

Administration of Therapeutic Agents) and/or Pharmaceutical Compounds

Administering therapeutic agent(s) according to the methods of the invention may include application of therapeutic agent(s) alone or formulating the therapeutic agent(s) with one or more of the compounds described supra as pharmaceutical compositions and administering the pharmaceutical compositions to a mammalian subject or host, including a human patient, intranasally to the upper third of the nasal cavity. The therapeutic agent(s) and/other components of the pharmaceutical composition thereof, e.g., vasoconstrictor, delivery-enhancement agent and/or antibiotic may be administered at one of a variety of doses sufficient to provide an effective amount at the desired point of action of the therapeutic agent(s) and/or pharmaceutical composition component. Doses for humans and other mammals can range from about 0.001 mg/kg to about 100 mg/kg, preferably from about 0.01 mg/kg to about 10 mg/kg, preferably from about 0.1 mg/kg to about 1-10 mg/kg. As noted, vasoconstrictor(s), delivery-enhancement agent(s), and/or antibiotic(s) may be delivered as pre-treatment, co-treatment and/or post-treatment with the therapeutic agent(s) and/or pharmaceutical composition, either alone or as a component of the pharmaceutical composition.

For application to the upper third of the nasal cavity as suspensions, aerosols, sprays or drops, the therapeutic agent(s) and/or pharmaceutical composition(s) can be prepared according to techniques well known in the art of pharmaceutical formulation. The compositions can be prepared as suspensions of the agent(s) in solutions which may comprise salts such as saline, components such as phosphate, succinate or citrate buffers to maintain pH, osmoregulatory and osmotic agents such as taurine, and suitable preservatives, absorption promoters to enhance bioavailability, fluorocarbons or other solubilizing or dispersing agents known in the art. The means of applying a pharmaceutical composition intranasally to the upper third of the nasal cavity may be in a variety of forms such as a powder, spray, gel or nose drops.

Other forms of compositions for administration of therapeutic agent(s) and/or pharmaceutical compositions or elements thereof include a suspension of a particulate, such as an emulsion, a liposome, or in a sustained-release form to prolong the presence of the pharmaceutically active agent in an individual. The powder or granular forms of the pharmaceutical composition may be combined with a solution and with a diluting, dispersing or surface-active agent. Additional compositions for administration include a bioadhesive to retain the agent at the site of administration at the upper third of the nasal cavity, for example a spray, paint, or swab applied to the mucosa. A bioadhesive can refer to hydrophilic polymers, natural or synthetic, which, by the hydrophilic designation, can be either water soluble or swellable and which are compatible with the pharmaceutical composition. Such adhesives function for adhering the formulations to the mucosal tissues of the upper third of the nasal cavity. Such adhesives can include, but are not limited to, hydroxypropyl cellulose, hydroxypropyl methylcellulose, hydroxy ethylcellulose, ethylcellulose, carboxymethyl cellulose, dextran, gaur gum, polyvinyl pyrrolidone, pectins, starches, gelatin, casein, acrylic acid polymers, polymers of acrylic acid esters, acrylic acid copolymers, vinyl polymers, vinyl copolymers, polymers of vinyl alcohols, alkoxy polymers, polyethylene oxide polymers, polyethers, and combinations thereof. The composition can also be in the form of lyophilized powder, which can be converted into solution, suspension, or emulsion before administration. The pharmaceutical composition is preferably sterilized by membrane filtration and is stored in unit-dose or multi-dose containers such as sealed vials or ampoules.

The pharmaceutical composition may be formulated in a sustained-release form to prolong the presence of the active therapeutic agent(s) in the treated individual. Many methods of preparation of a sustained-release formulation are known in the art and are disclosed in Remington's Pharmaceutical Sciences. Generally, the therapeutic agent(s), pharmaceutical composition and/or components of the pharmaceutical composition, i.e., vasoconstrictor, delivery-enhancement agent and/or antibiotic may be entrapped in semi-permeable matrices of solid hydrophobic polymers. The matrices can be shaped into films or microcapsules. Matrices can include, but are not limited to, polyesters, co-polymers of L-glutamic acid and gamma ethyl-L-glutamate, polylactides, polylactate polyglycolate, hydrogels, non-degradable ethylene-vinyl acetate, degradable lactic acid-glycolic acid copolymers, hyaluronic acid gels, and alginic acid suspensions. Suitable microcapsules can also include hydroxymethylcellulose or gelatin and poly-methyl methacrylate. Microemulsions or colloidal drug delivery systems such as liposomes and albumin microspheres can also be used.

### Delivery Systems

Therapeutic agent(s) and/or a pharmaceutical composition comprising at least one therapeutic agent and/or components of the pharmaceutical composition of the present invention may further be dispensed and applied to the upper third of the nasal cavity as a powdered or liquid nasal spray, suspension, nose drops, a gel, film or ointment, through a tube or catheter, by syringe, by packtail, by pledget (a small flat absorbent pad), by nasal tampon or by submucosal infusion. In some aspects of the present invention, the methods comprise administering to an individual therapeutic agent(s) and/or a pharmaceutical composition thereof to the upper third of the nasal cavity by way of a delivery device. Nasal drug delivery can be carried out using devices including, but not limited to, unit dose containers, pump sprays, droppers, squeeze bottles, airless and preservative-free sprays, nebulizers (devices used to change liquid medication to an aerosol particulate form), metered dose inhalers, and pressurized metered dose inhalers. In some aspects, an accurate effective dosage amount is contained within a bioadhesive patch that is placed directly within and on the upper third of a nasal cavity.

Therapeutic agent(s) and/or a pharmaceutical composition comprising at least one therapeutic agent and/or components of the therapeutic composition of the present invention may be conveniently delivered to the upper third of the nasal cavity in the form of an aerosol spray using a pressurized pack or a nebulizer and a suitable propellant including, but not limited to, dichlorodifluoromethane, trichlorofluoromethane, dichlorotetrafluoroethane, hydrocarbons, compressed air, nitrogen or carbon dioxide. An aerosol system requires the propellant to be inert towards the therapeutic agent(s) and/or pharmaceutical composition as will be readily recognized by the skilled artisan. In the case of a pressurized aerosol, the dosage unit may be controlled by providing a valve to deliver an accurately metered amount.

The means to deliver therapeutic agent(s) or pharmaceutical composition comprising at least one therapeutic agent and/or components of the pharmaceutical composition of the present invention to the upper third of the nasal cavity as a powder may be in a form such as microspheres delivered by a nasal insufflator device (a device to blow a gas, powder, or vapor into a cavity of the body) or pressurized aerosol canister. The insufflator produces a finely divided cloud of the dry powder or microspheres. The insufflator may be provided with means to ensure administration of a substantially metered amount of the pharmaceutical composition. The powder or microspheres should be administered in a dry, air-dispensable form. The powder or microspheres may be used directly with an insufflator which is provided with a bottle or container for the powder or microspheres. Alternatively the powder or microspheres may be filled into a capsule such as a gelatin capsule, or other single dose device adapted for nasal administration. The insufflator can have means such as a needle to break open the capsule or other device to provide holes through which jets of the powdery composition can be delivered to the upper third of the nasal cavity.

### Intermittent and Cyclic Dosing

In various embodiments of the invention, therapeutic agent(s) and/or a pharmaceutical composition comprising an effective amount of the at least one therapeutic agent and/or the components of the pharmaceutical composition may be administered as a single and one-time dose, or alternatively therapeutic agent(s) and/or the components of the pharmaceutical composition may be administered more than once and intermittently. By “intermittent administration” is intended administration of an effective amount of therapeutic agent(s) and/or the components of the pharmaceutical composition, followed by a time period of discontinuance, which is then followed by another administration of an effective amount, and so forth. Administration of the effective amount of therapeutic agent(s) and/or the components of the pharmaceutical composition may be achieved in a continuous manner, as for example with a sustained-release formulation, or it may be achieved according to a desired daily dosage regimen, as for example with one, two, three, or more administrations per day. By “time period of discontinuance” is intended a discontinuing of the continuous sustained-released or daily administration of the therapeutic agent(s) and/or the components of the pharmaceutical composition. The time period of discontinuance may be longer or shorter than the period of continuous sustained-release or daily administration. During the time period of discontinuance, the therapeutic agent(s) and/or the components of the pharmaceutical composition level in the relevant tissue is substantially below the maximum level obtained during the treatment. The preferred length of the discontinuance period depends on the concentration of the effective dose and the form of therapeutic agent(s) and/or the components of the pharmaceutical composition used. The discontinuance period can be at least 2 days, preferably is at least 4 days, more preferably is at least 1 week and generally does not exceed a period of 4 weeks. When a sustained-release formulation is used, the discontinuance period must be extended to account for the greater residence time of the at least one therapeutic agent at the site of injury. Alternatively, the frequency of administration of the effective dose of the sustained-release formulation can be decreased accordingly. An intermittent schedule of administration of therapeutic agent(s) and/or the components of the pharmaceutical composition may continue until the desired therapeutic effect, and ultimately treatment of the disease or disorder is achieved.

In yet another embodiment, intermittent administration of the effective amount(s) of therapeutic agent(s) and/or the components of the pharmaceutical composition is cyclic. By “cyclic” is intended intermittent administration accompanied by breaks in the administration, with cycles ranging from about 1 month to about 2, 3, 4, 5, or 6 months. For example, the administration schedule might be intermittent administration of the effective dose of therapeutic agent(s) and/or the components of the pharmaceutical composition, wherein a single short-term dose is given once per week for 4 weeks, followed by a break in intermittent administration for a period of 3 months, followed by intermittent administration by administration of a single short-term dose given once per week for 4 weeks, followed by a break in intermittent administration for a period of 3 months, and so forth. As another example, a single short-term dose may be given once per week for 2 weeks, followed by a break in intermittent administration for a period of 1 month, followed by a single short-term dose given once per week for 2 weeks, followed by a break in intermittent administration for a period of 1 month, and so forth. A cyclic intermittent schedule of administration of therapeutic agent(s) and/or the components of the pharmaceutical composition to a subject may continue until the desired therapeutic effect, and ultimately treatment of the disorder or disease is achieved.

The invention has been described with reference to various specific and preferred embodiments and techniques. However, it should be understood that many variations and modifications may be made while remaining within the spirit and scope of the invention.

We claim:

1. A method for treating a first disease or condition of a mammal's damaged or degenerating or injured central nervous system, wherein the mammal has a second systemic, non-central nervous system disease or condition that contraindicates systemic administration for treatment of the first disease or condition, comprising:
applying at least one therapeutic agent to the upper third of the nasal cavity of the mammal;
enabling the at least one therapeutic agent to access the damaged central nervous system by bypassing the blood-brain barrier;
treating the first disease or condition of the mammal's central nervous system; and
protecting the mammal's second disease or condition by ensuring that the at least one therapeutic agent is not administered systemically to the mammal.

2. The method of claim 1, further comprising administering the at least one therapeutic agent to a tissue innervated by the olfactory nerve, wherein the at least one therapeutic agent bypasses the blood-brain barrier to access the damaged or degenerating or injured central nervous system to treat the first disease or condition of the mammal; and
protecting the mammal by minimizing systemic delivery of the at least one therapeutic agent outside of the central nervous system and, therefore minimizing exposure of the second disease or condition to the at least one therapeutic agent.

3. The method of claim 2, further comprising the at least one therapeutic agent bypassing the blood-brain barrier by migrating along a neural pathway into the damaged or degenerating or injured central nervous system to treat the second disease or condition of the mammal.

4. The method of claim 1, wherein the second disease or condition is selected from at least one of the group consisting of pregnancy, female mammals trying to become pregnant, and female mammals at risk of becoming pregnant.

5. The method of claim 1, wherein the second disease or condition comprises female mammals who are breast feeding.

6. The method of claim 1, wherein the second disease or condition comprises liver damage.

7. The method of claim 6, wherein the mammal's liver damage is due to hepatitis C or alcoholism.

8. The method of claim 1, wherein the second disease or condition comprises an autoimmune disease.

9. The method of claim 8, wherein the autoimmune disease comprises HIV/AIDS.

10. The method of claim 1, wherein the second disease or condition comprises at least one stomach ulcer.

11. The method of claim 1, wherein the second disease or condition comprises cancer wherein the cancer contraindicates the systemic administration of at least one therapeutic agent for treatment of the first disease or condition.

12. The method of claim 11, wherein the cancer is selected from the group consisting of throat cancer, esophageal cancer, stomach cancer and liver cancer.

13. The method of claim 1, wherein the first disease or condition of the mammal's damaged or degenerating or injured central nervous system comprises Parkinson's disease.

14. The method of claim 1, wherein the first disease or condition of the mammal's damaged or degenerating or injured central nervous system comprises Alzheimer's disease.

15. The method of claim 1, wherein the first disease or condition of the mammal's damaged or degenerating or injured central nervous system comprises ischemia.

16. The method of claim 1, wherein the first disease or condition of the mammal's damaged or degenerating or injured central nervous system comprises seizures.

17. The method of claim 1, wherein the first disease or condition of the mammal's damaged or degenerating or injured central nervous system comprises epilepsy.

18. The method of claim 1, wherein the first disease or condition of the mammal's comprises neurodegeneration resulting from damage or degeneration or injury to the central nervous system.

19. The method of claim 1, wherein the at least one therapeutic agent comprises at least one metal chelator.

20. The method of claim 1, further comprising pretreating the mammal's nasal cavity with at least one vasoconstrictor before applying the at least one therapeutic agent to the upper third of the mammal's nasal cavity.

21. The method of claim 1, further providing a pharmaceutical composition comprising the at least one therapeutic agent and an effective amount of at least one of the group consisting of a vasoconstrictor, a delivery enhancement agent, and an antibiotic;
applying an effective amount of the pharmaceutical composition to the upper third of the mammal's nasal cavity, thereby enabling the effective amount of the pharmaceutical composition to bypass the blood-brain barrier and directly delivering the effective amount of the pharmaceutical composition directly to the patient's central nervous system to treat the first disease or condition.

22. The method of claim 21, wherein the at least one delivery-enhancement agent further comprises one of the group consisting of hyaluronidase, migration-inducing activity and neuregulin.

23. The method of claim 21, further comprising pretreating the upper third of the nasal cavity with at least one antibiotic in an effective amount before applying the pharmaceutical composition.
